(19) United States
(12) Patent Application Publication
(10) Pub. No.: US 2017/0021075 A1
(43) Pub. Date: Jan. 26, 2017

(54) MID-DILUTION HEMODIAFILTRATION WITH MULTI-LINE DIALYSATE SUPPLY

(71) Applicant: Fresenius Medical Care Holdings, Inc., Waltham, MA (US)

(72) Inventors: Matthew Doyle, Concord, CA (US); Eric Hoffstetter, Pleasanton, CA (US)

(21) Appl. No.: 14/804,735

(22) Filed: Jul. 21, 2015

(51) Int. Cl. A61M 1/16

(52) U.S. Cl. CPC A61M 1/1601; A61M 2205/3331

(57) ABSTRACT

A hemodiafiltration system in accordance with an embodiment of the described system at least two dialyzers for hemodiafiltration, at least one dialysate supply, a sterility filter for generating a sterile substitution fluid, and a control unit which controls fluid (e.g., dialysate, substitution fluid, and blood) inputs and outputs to and from each of the at least two dialyzers, the at least one sterility filter, and the dialysis machine. The hemodiafiltration system described herein is capable of executing blood processing with enhanced clearance of small, middle, and large molecules using features that include independently supplying dialysate to multiple dialyzers.

### TECHNICAL FIELD

This patent application relates to blood cleansing and, more particularly, to mid-dilution hemodiafiltration systems.

### BACKGROUND

Hemodialysis is a process which employs a machine that includes a dialyzer to aid patients whose renal function has deteriorated to the point where their body cannot adequately rid itself of toxins. The dialyzer may include a semi-permeable membrane, the membrane serving to divide the dialyzer into two chambers. Blood is pumped through one chamber and a dialysis solution through the second. As the blood flows by the dialysis fluid, impurities, such as urea and creatinine, diffuse through the semi-permeable membrane into the dialysis solution. Dialysis treatment requires monitoring of several patient vital signs and dialysis parameters during the dialysis process in order to optimize the overall efficacy of the dialysis procedure. Some examples of parameters monitored and analyzed by a dialysis machine or equipment include the blood access flow rate or the rate at which blood flows out of the patient to the dialyzer, a critical parameter; and the ratio Kt/V to measure dialysis efficiency, where K is the clearance or dialysance (both terms representing the purification efficiency of the dialyzer), t is treatment time and V is the patient's total water value.

Other purification techniques and processes may additionally be used. One such example is hemodiafiltration (HDF), which combines standard dialysis and hemofiltration into one process, whereby convective and diffusive clearance are achieved through the use of substitution fluid. The removal of uremic toxins by diffusion is accomplished by establishing a concentration gradient, whereas convective clearance is achieved through continuous addition of substitution fluid added to the blood (either prior to the dialyzer (pre-dilution) or after the dialyzer (post-dilution)) with an equal amount ultrafiltered across the dialyzer carrying with it uremic toxins.

Substitution fluid also may be added between two dialyzers, or between the first and second stage of a two stage hemofilter. Such methods of hemodiafiltration are known as mid-dilution hemodiafiltration. One such example is the OLpūr MD Series Hemodiafilter from Nephros (see, e.g., U.S. Pat. No. 6,406,631, U.S. Pat. No. 6,303,036 and U.S. Pat. No. 6,423,231, which are all incorporated herein by reference).

Substitution fluid may be generated by on-line filtration of a non-sterile dialysate through a suitable filter cartridge rendering it sterile and non-pyrogenic. Such online production of substitution fluid is described, inter alia, in D. Limido et al., “Clinical Evaluation of AK-100 ULTRA for Predilution HF with On-Line Prepared Bicarbonate Substitution Fluid. Comparison with HD and Acetate Postdilution HF,” International Journal of Artificial Organs, Vol. 20, No. 3 (1997), pp. 153-157.

Typically one dialyzer cartridge containing a high flux semi-permeable membrane is used for hemodialysis and hemodiafiltration as additional filters and bloodlines add cost; however, more complex methods of hemodiafiltration exist in which two or more dialyzers may be used in series. Such set-ups are described in J. H. Miller et al., “Technical Aspects of High-Flux Hemodiafiltration for Adequate Short (Under 2 Hours) Treatment,” Transactions of American Society of Artificial Internal Organs (1984), pp 377-380. In these set-ups, counter-current flow of dialysate is supplied to more than one dialyzer with simultaneous ultrafiltration in the first dialyzer.

When two dialyzers are used, mid-dilution hemodiafiltration may be accomplished without the necessity of a specific dialyzer filter suitable for mid-dilution hemodiafiltration. For various descriptions of more recent multidialyzer mid-dilution systems, reference is made, for example, to: U.S. Pat. No. 8,029,454 B2 to Kelly et al., entitled “High Convection Home Hemodialysis/Hemofiltration and Sorbent System,” US 2011/0098625 A1 to Masala et al., entitled “Hemodialysis or hemo(dia)filtration apparatus and a method for controlling a hemodialysis or hemo(dia)filtration apparatus,” EP 0951303 B1 to Polaschegg, Hans-Dietrich entitled “Device for Preparation of Substitution Solution,” and U.S. Pat. No. 7,067,060 to Collins et al., entitled “Ionic Enhanced Dialysis/Diafiltration System,” which are all incorporated herein by reference.

There are numerous benefits to mid-dilution with respect to pre-dilution and post-dilution hemodiafiltration. U.S. Pat. No. 6,423,231 to Collins et al, “Non-Isosmotic Diafiltration Systems,” which is incorporated herein by reference, notes the following rationale for mid-dilution hemodiafiltration:
“An advantage of this process is that a gain in clearance of small molecular weight substances in the first dialyzer overshadows a loss in clearance of small molecular weight substances due to the dilution of blood concentration entering the second dialyzer. Further, clearance of larger molecular weight substances is enhanced considerably, because the total filtration level of plasma water is practically doubled (e.g. 40% to 80% of the incoming blood flow rate may be filtered) compared to filtration using a single dialyzer operating in a postdilution mode.”

Substitution fluid can also be added simultaneously before and after the dialyzer, resulting in a process known as mixed dilution hemodiafiltration. Examples of such therapies are seen in the study by Feliciani et al., “New strategies in haemodiafiltration (HDF): prospective comparative analysis between on-line mixed HDF and mid-dilution HDF,” Nephrol Dial Transplant (2007) 22: 1672-1679. The advantage of mixed-dilution hemodiafiltration is that it can be performed without the limitations of a dual stage hemofilter as noted in the study:
“In mixed HDF, as in the present experimental setting, any TMP increase was prevented by the feedback TMP regulation through repeated small shifts of infusion fluid from the postdilution to the pre-dilution site with no effect on the total infusion and ultrafiltration rate . . . . On the contrary, in mid-dilution HDF, very high pressure values were recorded right from the beginning of the sessions both at the inlet blood compartment and along its first part, the post-dilution section, up to the infusion site. As sessions progressed, further increase occurred and inlet blood pressure (PB in) and infusion pressure (P mid) rose to dangerous levels despite attempts to reduce this effect with repeated manual reduction of the total infusion rate.”

The pressure limitations noted in the above-identified study can be reduced for current mid-dilution hemodiafiltration methods that use two dialyzers, because pore size less restricted, and can thus be increased, when selecting from dialyzers instead of two stage hemofilters. Moreover, the volume entering the first and second dialyzer would be approximately doubled, because the full amount of dialyzer fibers would be used rather than only the first stage of dialyzer fibers shared in the two stage hemofilter. The limitations described by Feliciani et al., however, would extend to mid-dilution methods utilizing two dialyzers as well. The reason for this is that pre-dilution in the first dialyzer is not possible in a mid-dilution scheme, and as the therapy progresses and ultrafiltrate is removed, the increase in transmembrane pressure resultant of hemoconcentration cannot be overcome.

In addition to pressure issues related to current methods of mid-dilution hemodiafiltration, when multiple dialyzers are currently used to perform mid-dilution hemodiafiltration, clearance is limited by the method through which dialysate is introduced. The methods implemented by the prior art supply dialysate either countercurrent to the blood flow by entering into the second dialyzer, exiting the second dialyzer, and then entering and exiting the first dialyzer, as is shown in for example in U.S. Pat. No. 7,067,060 to Collins et al., which is incorporated herein by reference.

Dialysate can also be introduced concurrent to blood flow by entering into the first dialyzer, exiting the first dialyzer, and then entering and exiting the second dialyzer as is shown, for example, in U.S. Pat. No. 8,029,454 B2 to Kelly et al., entitled “High Convection Home Hemodialysis/Hemofiltration and Sorbent System,” which is incorporated herein by reference. The dialysate supplied to each of the two dialyzers is not independently controlled in such a system. Furthermore, the method of this system does not provide a reliable procedure to address hemoconcentration in the first dialyzer, as would be ameliorated by pre-dilution and mixed-dilution hemodiafiltration methods.

Accordingly, it would be desirable provide a hemodiafiltration method and device that are capable of executing blood processing with enhanced clearance of small, middle, and large molecules and that ameliorate the problems associated with high dialyzer hemoconcentrations in the first stage of dual stage hemofilters or the first dialyzer in dual dialyzer mid-dilution hemodiafiltration methods. Additionally, it would be desirable to provide a mid-dilution hemodiafiltration method that is not limited by hemoconcentration and can additionally provide other advantages, including enhanced uremic and large/middle molecule clearance of uremic toxins, such as Beta-2-microglobulin, and beneficially addressing secondary membrane formation.

### SUMMARY

According to the system described herein, a hemodiafiltration device includes at least two dialyzers for performing hemodiafiltration. At least one dialysate supply is provided for supplying dialysate. A sterility filter is provided for generating a sterile substitution fluid. A control unit controls medical fluid inputs and outputs to and from each of the at least two dialyzers, wherein the control unit controls the supply of dialysate such that the dialysate is independently supplied to the at least two dialyzers from the at least one dialysate supply. The dialysate supply may include a plurality of dialysate supply lines and/or a plurality of dialysate return lines. The medical fluid may include the dialysate, the substitution fluid and/or blood. At least one pump may be provided that is configured to pump the medical fluid, and the pump may be controlled by the control unit. The control unit may include at least one specific processor configured to control the hemodiafiltration device and that executes executable code stored on a non-transitory computer-readable medium.

According further to the system described herein, a method for performing hemodiafiltration includes performing hemodiafiltration using at least two dialyzers. Dialysate is supplied from at least one dialysate supply. A sterile substation fluid is generated using a sterility filter. Medical fluid inputs and outputs to and from each of the at least two dialyzers are controlled using a control unit, and in which the supply of dialysate is controlled such that the dialysate is independently supplied to the at least two dialyzers from the at least one dialysate supply. The dialysate supply may include a plurality of dialysate supply lines and/or a plurality of dialysate return lines. The medical fluid may include the dialysate, the substitution fluid and/or blood. At least one pump may be provided that is configured to pump the medical fluid, and the pump may be controlled by the control unit. The control unit may include at least one specific processor configured to control the hemodiafiltration device and that executes executable code stored on a non-transitory computer-readable medium.

According further to the system described herein, a system for processing medical fluid includes a hemodiafiltration device and at least one component for transporting medical fluid to or from the hemodiafiltration device. The hemodiafiltration device includes at least two dialyzers for performing hemodiafiltration. At least one dialysate supply is provided for supplying dialysate. A sterility filter is provided for generating a sterile substitution fluid. A control unit is provided which controls medical fluid inputs and outputs to and from each of the at least two dialyzers. The control unit controls the supply of dialysate such that the dialysate is independently supplied to the at least two dialyzers from the at least one dialysate supply. The dialysate supply may include a plurality of dialysate supply lines and/or a plurality of dialysate return lines. The medical fluid may include the dialysate, the substitution fluid and/or blood. At least one pump may be provided that is configured to pump the medical fluid, and the pump may be controlled by the control unit. The control unit may include at least one specific processor configured to control the hemodiafiltration device and that executes executable code stored on a non-transitory computer-readable medium.

### BRIEF DESCRIPTION OF THE DRAWINGS

Embodiments of the system described herein are explained with reference to the several figures of the drawings, which are briefly described as follows.

FIG. 1 is a schematic illustration of a blood circuit of a hemodiafiltration device system in accordance with an embodiment of the system described herein.

FIG. 2 is a schematic illustration of a hemodiafiltration device system in accordance with an embodiment of the system described herein.

FIG. 3 is schematic illustration of a hemodiafiltration device system in accordance with an embodiment of the system described herein.

FIG. 4 is schematic illustration of a hemodiafiltration device system in accordance with an embodiment of the system described herein.

FIG. 5 is a flow diagram showing process of a control unit for monitoring and controlling the operation of the hemodiafiltration system in accordance with an embodiment of the system described herein.

### DETAILED DESCRIPTION OF VARIOUS EMBODIMENTS

A hemodiafiltration system in accordance with an embodiment of the system described herein includes at least two dialyzers for hemodiafiltration, at least one sterility filter for generating a sterile substitution fluid, a dialysate supply, and a control unit which controls fluid (dialysate, substitution fluid, and blood) inputs and outputs to and from each of the at least two dialyzers, the at least one sterility filter, and the dialysis machine. The hemodiafiltration system described herein is capable of executing blood processing with enhanced clearance of small, middle, and large molecules using features that include independently supplying dialysate to multiple dialyzers. The exemplary embodiments discussed herein provide dialysate to two dialyzers. However, it is noted that the system described herein may be applied to more than two dialyzers, in which case, each additional dialyzer adds the need for an additional valve and transmembrane pressure (TMP) sensor, as further discussed elsewhere herein. Additionally, it should be appreciated that the hemodiafiltration system in accordance with an embodiment of the system described herein would continue to have the ability to perform standard hemodialysis therapies, if desired. Moreover, it should be appreciated that the hemodiafiltration system in accordance with an embodiment of the system described herein would have the ability to perform pre and post-dilution hemodiafiltration therapies.

The dialyzers may contain a semi-permeable membrane and, in an embodiment of the system described herein, may be aligned in a series in which substitution fluid produced by the at least one sterility filter is introduced between the first and second dialyzer. The control unit may contain various pumps, pressure monitoring devices, valves, electronic components, connector fittings, tubing, etc., as required in order to coordinate the operation of the other system components.

Blood enters the bloodside compartment of the first dialyzer, whereby ultrafiltration on the first dialyzer results in plasma water being filtered across the semi-permeable membrane into the adjacent dialysate compartment. As the blood leaves the first dialyzer, substitution fluid is added to the blood and the diluted blood then enters the bloodside compartment of the second dialyzer, whereby ultrafiltration in the second dialyzer will result in plasma water being filtered across the semi-permeable membrane into the adjacent dialysate compartment. In this manner, the substitution fluid acts as a post-dilution fluid relative to the first dialyzer as well as a pre-dilution fluid relative to the second dialyzer.

To ensure that blood does not become diluted or over-concentrated as it passes through each dialyzer, a fluid balancing system and a separate UF pump are used. In accordance with an embodiment of the system described herein, the dialysate fluid, after being generated by a fluid balancing system, is partitioned through use of valves (either individual or three way) wherein a fraction of the total fluid enters the first dialyzer and runs parallel to the blood flow direction and exits to a spent dialysate line connected in parallel to the spent dialysate line of the second dialyzer, a fraction of the total fluid enters the second dialyzer and runs parallel to the blood flow direction and exits to a spent dialysate line connected in parallel to the spent dialysate line of the first dialyzer, and a fraction of the total fluid enters an at least one sterility filter for generating a sterile substitution fluid that is introduced between the first and second dialyzer at a controlled rate through use of a substitution fluid pump and exits to the spent dialysate line connected to the second dialyzer and/or exits to a spent dialysate line connected in parallel to the spent dialysate line of the first dialyzer. Through use of fresh dialysate at each dialyzer, the concentration gradient for diffusion is maximized. Pressures may be monitored both on the bloodside and the dialysate side of each dialyzer cartridge as a way to determine transmembrane pressure (TMP) across each dialyzer.

The spent dialysate from the first and second dialyzer is transported back to the dialysis machine. The UF Pump will generate convective clearance on both the first and second dialyzer; depending on if the valve dedicated to the spent dialysate line associated with said dialyzer is open or closed. Through use of valve duty cycling on the fresh dialysate valves associated with each fresh dialysate line, the amount of fresh dialysate supplied to the first and second dialyzer can be controlled. Additionally, through use of valve duty cycling on the spent dialysate valves, the amount of fluid removed from each dialyzer can be controlled.

Through use of this duty cycling, hemoconcentration issues typically associated with mid-dilution hemodiafiltration can be eliminated. As the treatment progresses and ultrafiltrate is removed, pressure issues associated with hemoconcentration will begin to be detectable in the first dialyzer through changes in transmembrane pressure. When this is detected, the duty-cycling of the fresh dialysate valves, and/or the spent dialysate valves will adjust using a combination of the following two methods: 1) The amount of fresh dialysate supplied to the first dialyzer shall be increased, resulting in a decrease of dialysate flow to the second dialyzer by an amount equal to said increase, 2) The amount of spent dialysate removed from the first dialyzer will be decreased, resulting in an increase of dialysate removal from the second dialyzer. In so doing, ultrafiltration rates can remain constant to support substitution fluid addition throughout the duration of the treatment.

It is noted that some combination of these two methods may be used depending on the measured transmembrane pressure of the first and second dialyzer. Additionally, the control system may be optimized using clearance calculations, such as the Michaels' equations as discussed in “Operating parameters and performance criteria for hemodialyzers and other membrane-separation devices,” Trans Am Soc Artif Int Organs 7: 387-392, 1966, and discussed further in the 3rd Edition of Replacement of Renal Function by Dialysis: a textbook of dialysis.

A dialysis machine according to the system described herein is thereby provided which is adapted to perform improved hemodiafiltration. The dialysis machine may be adapted through utilization of a Hemodiafiltration Module bay along with the required hydraulic changes. In this way, existing hemodialysis machines, such as the Fresenius 2008T machine, may be upgraded in the field to execute the improved therapy described herein. Such upgraded machines would continue to have the ability to perform standard hemodialysis therapies, if desired. Alternatively, the hemodiafiltration device of the system described herein may be embodied in an “add-on” system which may be used in conjunction with a standard UF controlled dialysis, machine to performed improved hemodiafiltration.

The hemodiafiltration method and device of the system described herein is principally described herein in the context of a stand-alone dialysis/hemodiafiltration machine. However, it is noted that the system described herein may be appropriately used in connection with other hemodialysis based techniques and modalities. In an embodiment of the system described herein, as described in more detail below with reference to the drawings, the hemodiafiltration device includes a first and a second dialyzer. The hemodiafiltration device includes at least one sterility filter, which may contain semi-permeable membranes for removing bacteria, endotoxins, and other particulate from the dialysate to generate suitable substitution fluid. The extracorporeal blood circuit may contain various pumps, pressure monitoring devices, valves, electronic components, connector fittings, tubing, etc., as required.

Preparation of dialysate solution includes mixing of water with dialysate concentrates. Water is generated using a suitable method of pre-treatment (e.g., Reverse Osmosis). The dialysate fluid generated from the balancing chamber is partitioned, through use of valves and valve duty-cycling, for three sources: 1) the first dialysate of the first dialyzer, 2) the fresh dialysate of the second dialyzer, and 3) the substitution fluid. After being partitioned, fresh dialysate enters both the first and second dialyzer, independently and concurrently, and runs parallel to the blood flow direction. The dialysate fluid acts to provide a concentration gradient against the bloodside fluid thereby facilitating diffusion of solutes across the semipermeable membrane. Upon exiting the first and/or second dialyzer, spent dialysate fluid is transported back to the hemodiafiltration device.

The sterile/non-pyrogenic substitution fluid for use in conjunction with the system described herein is prepared by drawing a portion of fresh dialysate solution from the dialysate inlet line and pumping it through a sterile filter cartridge. Through use of an additional sterile filter for the dialysate, the substitution fluid is effectively double filtered before introduction into the blood stream. The dialysis machine used in conjunction with the system described herein may perform all of its normal functions, such as monitoring flowrates and pressures, controlling net ultrafiltration, monitoring used dialysate for blood presence, etc. The hemodiafiltration device of the system described herein operates in conjunction with the dialysis machine, as part of the dialysis machine or as an add-on module. The fluid handling components of the hemodiafiltration system may be integrated with a microprocessor unit for controlling and executing the hemodiafiltration aspect of the treatment, or a control unit of the dialysis machine may be adapted to control the hemodiafiltration aspects of the treatment. Additionally, it should be appreciated that the hemodiafiltration device of the system described herein would also be capable of performing standard hemodialysis therapies, pre-dilution hemodiafiltration therapies, and post-dilution hemodiafiltration therapies using one dialyzer, if desired.

In this case of using one dialyzer, one fresh and one spent dialysate line would provide for supplying dialysate and ultrafiltration, and the unused fresh and spent dialysate lines would remain attached to a dialysate line shunt. The electronics, e.g., a control unit, associated with the dialysate line shunt would detect that not all dialysate lines were in operation and, in conjunction with the control unit, ensure that dialysate is only supplied to the dialysate line in use. Additionally, the machine may provide an informational message on the treatment display to confirm the desired mode of machine operation. As detected by the control unit, if standard hemodialysis bloodlines were attached to the machine, substitution fluid would not be generated and a hemodialysis therapy could be performed. If bloodlines capable of performing pre-dilution or post-dilution hemodiafiltration were attached to the machine, substitution fluid would be generated and a hemodiafiltration therapy could be performed.

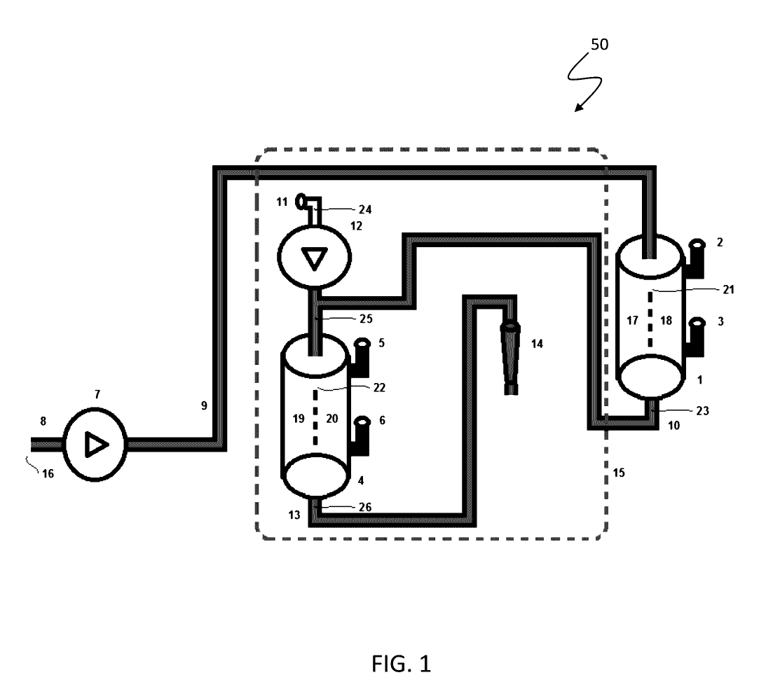

FIG. 1 schematically illustrates a hemodiafiltration device blood circuit 50 in accordance with an embodiment of the system described herein. It should be appreciated that system of FIG. 1 demonstrates only one embodiment of the system described herein, and that other possible configurations of the system of the system described herein may be equally or even more suitable, depending on specific requirements.

In the circuit 50 of FIG. 1 blood to be cleaned 16 enters the pre-pump portion of the arterial blood line 8 via blood pump 7 and enters the post-pump portion of the arterial blood line 9. The blood then enters a first dialyzer 1 after passing through blood flow and/or blood pressure monitoring devices (not shown) which send data to a control unit (not shown). The blood is carried by suitable tubing, for example, bloodline tubing made from flexible polyvinylchloride (PVC). The flowrate of incoming blood is generally in the range of 100 to 600 ml/min, preferably 200 to 500 ml/min.

The first dialyzer 1 contains a semi-permeable membrane 21 that divides the dialyzer in a blood side compartment 17 and a dialysate compartment 18. As blood 16 passes through the blood compartment 17, plasma water containing blood substances is filtered across semi-permeable membrane 21. Fresh dialysate is supplied to the first dialyzer from dialysate line 2, and spent dialysate is removed from the first dialyzer from dialysate line 3. Additional blood substances are also transferred across semi-permeable membrane 21 by diffusion due to a difference in concentration between the blood compartment 17 and the dialysate compartment 18. The dialyzer cartridge may be of any type suitable for hemodialysis, hemodiafiltration, or hemofiltration, for example, the Fresenius F200NR Optiflux, available from Fresenius Medical Care, Waltham, Mass. The membrane 21 may be a low to high flux membrane.

Blood exiting dialyzer 1 (denoted 23) enters intermediate tubing set 10 and is mixed with sterile substitution fluid 24 supplied from hemodiafiltration port 11 through substitution fluid pump 12 to form a blood/substitution fluid mixture 25. This mixture enters a second dialyzer 4 containing a semi permeable membrane 22 which divides the second dialyzer 4 into a blood compartment 19 and a dialysate compartment 20.

As the blood/substitution fluid mixture 25 passes through the blood compartment 19, plasma water containing blood substances is filtered across the semi-permeable membrane 22. As in the first dialyzer cartridge, additional blood substances are transferred across semi-permeable membrane 22 by diffusion due to concentration gradients between the blood and dialysate compartments. Fresh dialysate is supplied to the second dialyzer from dialysate line 5, and spent dialysate is removed from the second dialyzer by dialysate line 6. Cleansed blood 26 exits second dialyzer 4, enter venous tubing 13 and then enters a venous drip chamber 14 with associated blood pressure monitoring devices (not shown) which send this pressure data to a control unit (not shown), and is then returned to the patient (not shown) through suitable tubing, for example, bloodline PVC tubing, as is known in the art. The second dialyzer cartridge, like the first dialyzer, may be of any suitable type as described above.

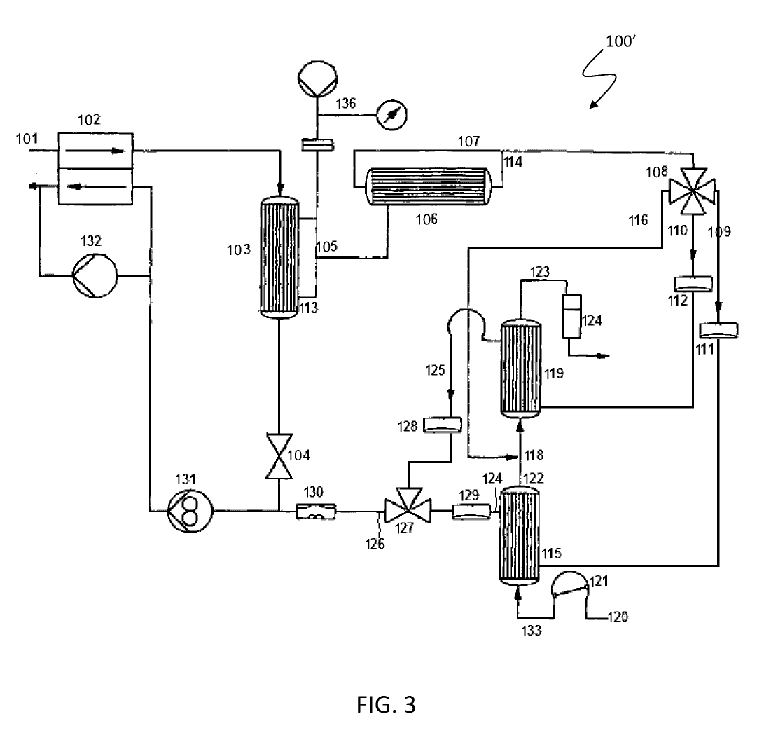

Module bay 15 is indicated to illustrate that such a machine adaptation could be implemented through use of a machine module bay on existing hemodialysis machines such as the 2008T machine by the company Fresenius Medical Care. Implementation of such a module bay would require hydraulic changes, as is discussed in further detail below with respect to FIG. 3.

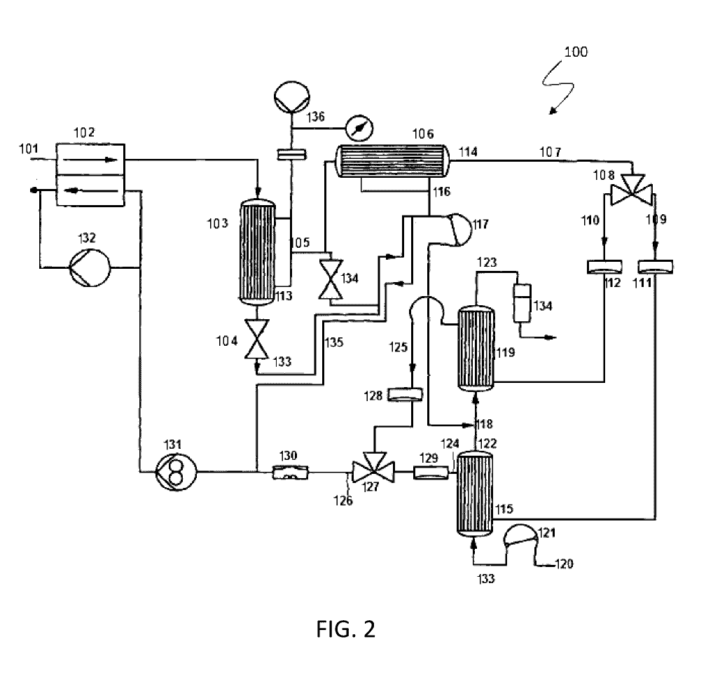

FIG. 2 schematically illustrates a hemodiafiltration device 100 in accordance with an embodiment of the system described herein. The dialysate solution used for the system described herein may be prepared as follows. A suitable quality of water, such as reverse osmosis water as is known in the art, is provided from a water source (not shown). The water enters a water preparation module (not shown) that heats and degasses the water. Any suitable known heating and degassing module may be used in conjunction with the system described herein. Examples of such modules are included in the following systems: the Baxter SPS1550, available from Baxter Health Care, Deerfield, Ill.; the Cobe Centry System 3, available from Cobe Labs, Lakewood, Colo.; the Fresenius A2008, available from Fresenius Medical Care, Waltham, Mass.; and the Althin System 1000, available from Althin Medical, Miami, Fla. The degassed, heated water feeds is proportioned with acid and bicarbonate, as is known in the art, to generate fresh dialysate fluid. The fresh dialysate fluid 101 enters into balancing system 102, to ensure that the inlet and outlet amounts are equal. Examples of such balancing systems can be seen in the 2008 or 4008 by the company Fresenius Medical Care, the machine Centry 3 of company Cobe, the machine System 1000 of company Althin Medical, the machine MIRO-CLAV of company Baxter, or the machine DIALOG of company B. Braun-Melsungen.

The fresh dialysate fluid 101 from the balancing system 102 passes through a conductivity and temperature monitor (not shown) which prevent incorrect dialysate fluid composition and/or temperature from reaching the patient, and then through a first sterile filter 103 comprising a semipermeable membrane.

When valve 104 is closed, the dialysis fluid 101 passes through the membrane of the sterile filter 103 to a line 105 for producing a cleansed dialysate fluid 113. From line 105, the cleansed dialysate fluid 113 passes through a second sterile filter 106 to a line 107. The dialysate fluid exiting the second sterile filter 106 (denoted 114) traverses the line 107 and enters into a three-way valve 108. The three-way valve 108 proportions the dialysate through use of software-duty cycling. Through use of duty-cycling of valves, or in other words toggling the valves off and on at known rates, a total amount of the dialysate fluid 114 can be accurately partitioned to a first line 109 and a second line 110. Such duty-cycling is discussed in further detail below. It should be appreciated that, instead of a three-way valve, two individual valves may also be used, if desired.

A fraction of the dialysate fluid 114 travels through both the lines 109 and 110 concurrently. The first and second dialysate line each have an associated pressure transducer 111 and 112 to assist with monitoring of transmembrane pressure. The dialysate fluid 114 from the first line 109 enters into a first dialyzer 115. When the valve 104 is opened, fluid bypasses the sterile filter to a line 133. Cleansed dialysate fluid 113 can also enter the line 133 through a valve 134 when it is opened. The line 133 connects to a hemodiafiltration module 117. A line 135 also connects to the hemodiafiltration module 117 and is downstream of line 133. Through use of the lines 133 and 135, dialysate can bypass the second sterile filter 106, first dialyzer 115, and second dialyzer 119 to enter balancing system 102 using a dialysate circulation pump 131 and further to the drain. An optional air pump 136 to perform tests of filter integrity known in the art can be included if it is desired to be able to test the integrity of the sterile filters and dialyzer filters.

A line 116 connects the hemodiafiltration module 117 between the second filter 106 and the mid-dilution injection point 118. The hemodiafiltration module 117 would comprise all the necessary valves (ex. Clamp Valve, Rinse Valve, Return Valve), additional electronics (Ex. Control boards, Interface Boards, Opto/Hall Port Sensors, etc.) necessary (not shown) for HDF pump modules known in the state of the art. Examples of such HDF pump modules include the Fresenius 4008 HDF Hemodialysis System among others.

Optionally, line 116 can include a flow sensor (not shown). The dialysis fluid also passes through the membrane of the second sterile filter 106 to produce a sterile substitution fluid in line 116 to be supplied to the blood in an extracorporeal circuit to be described in further detail below. As a result, the flow from the first sterile filter 103 is measured or controlled by the balancing unit 102 and then proportioned via the hemodiafiltration module 117 for substitution fluid flow via line 116 and the remaining dialysate fluid 114 flows via line 107 to be used as fresh dialysate for the first dialyzer 115 and second dialyzer 119. It is desired to control the flow of substitution flow to obtain the goals of the treatment and this is controlled via the substitution pump associated with hemodiafiltration module 117.

The spent dialysis fluid leaves the first dialyzer 115 through a line 124, and passes through a dialysate pressure monitor 129. Spent dialysate fluid leaves the second dialyzer 119 through a line 125, and passes through a dialysate pressure monitor 128. These two lines meet at three-way valve 127, and enter into a shared line 126. It should be appreciated that, instead of a three-way valve, two individual valves may also be used, if desired. Line 126 further comprises a blood leak detector 130. The spent dialysate passes through the balancing system 102 using a dialysate circulation pump 131 and further to the drain. After blood leak detector 130, the spent dialysate enters an air separation chamber (not shown), which makes possible the separation of air, since many balancing systems are disturbed by air. Parallel to the balancing system 102 there is a UF Pump 132 to remove ultrafiltrate. Via line 116 the filtered and sterile substitution fluid reaches the mid-dilution injection point 118 through use of the substitution pump 117.

The extracorporeal circuit comprises according to known techniques a blood pump 121, an arterial tube system 133, the blood portion of the dialyzers 115 and 119 and a venous tube system 123 incorporating the venous drip chamber 134. Additionally, the extracorporeal circuit comprises an intermediate tubing system 122 with mid-dilution injection point 118.

In this way, mid-dilution hemodiafiltration is achieved as blood 120 enters the first dialyzer 115, mixes with substitution fluid at injection point 118, enters the second dialyzer 119, and then is returned to the patient (not shown).

Preparation of a sterile substitution fluid is performed by filtration of a dialysate across at least two filter membranes with a molecular weight cut-off of not more than 40,000 Daltons; however, smaller molecular weight cut-offs approaching 5,000 Daltons can be used.

FIG. 3 schematically illustrates a hemodiafiltration device 100′ in accordance with an embodiment of the system described herein. FIG. 3 is an alternative embodiment to FIG. 2 where the positive pressure of the dialysate along with an additional valve is used to allow for substitution fluid introduction between the first dialyzer 115 and second dialyzer 119. All analogously numbered components to FIG. 2 function in a similar way aside from the differences noted below.

Substitution fluid line 116 in this embodiment is located downstream of line 107, and is directly attached to a 4-way valve 108. Through use of duty-cycling of valves, a total amount of dialysate fluid 114 can be accurately partitioned to a first line 109, a second line 110, and a substitution line 116. It should be appreciated that a four-way valve is preferred; however, any desired combination of valves could equally be used.

A limitation of this set-up is that all the dialysate fluid 114 passes directly through the second sterile filter 106, rather than only the substitution fluid as is the case in FIG. 2. Valve 104 allows for bypass of the second sterile filter 106 and first dialyzer filter 115 and second dialyzer filter 119 in an analogous way to FIG. 2.

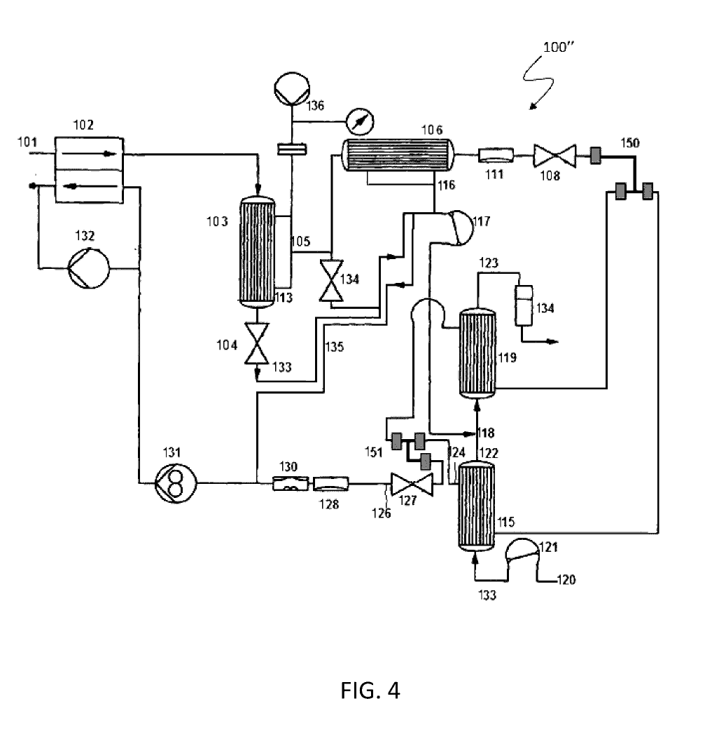

FIG. 4 schematically illustrates a hemodiafiltration device 100″ in accordance with an embodiment of the system described herein. FIG. 4 is an alternative embodiment to FIGS. 2 and 3. FIG. 4 is an alternative embodiment that may involve use of specific (1) Male Hansen Connector to (2) Female Hansen Connector lines. Simple examples are available, such as lines that are available from Molded Products, Inc; however, these lines do not allow for control of dialysate flowrates to each dialyzer, like that of the embodiments illustrated in FIGS. 1, 2, and 3 in connection with the system described herein.

Through reliance on such specific lines, hydraulic changes are minimized; however, through use of passive lines the amount of dialysate supplied to each dialyzer can no longer be controlled unless additional valves are incorporated into the passive lines. If additional valves are incorporated into lines 150 and 151, they would function in an analogous way to valves 108 and 127 respectively in FIG. 2.

Through use of passive lines 150 and 151 only one pressure monitor 111, rather than two pressure monitors, is required for each fresh dialysate line when compared to FIG. 2. Additionally, only one pressure monitor 128, rather than two pressure monitors, is required for each fresh dialysate line when compared to FIG. 2. All analogously numbered components to FIG. 2 function in a similar way. If additional valves are incorporated into lines 150 and 151, two pressure monitors would be required as illustrated in FIG. 2.

According to the system described herein, the fact that dialysate is independently supplied to each dialyzer provides further benefits. For example, the system described herein provides for the ability to address hemoconcentration without impacting the blood pump rate or significantly impacting clearance. By having independent dialysate lines according to the system described herein, the dialysate used to address hemoconcentration is occurring on the dialysate side and not the blood side, thereby aiding clearance by provide enhanced uremic and large/middle molecule clearance of uremic toxins, such as Beta-2-microglobulin. Moreover, blood pump rate does not need to be adjusted.

In particular, it is noted that with mid-dilution techniques, substitution fluid rates may eventually have to be reduced in known mid-dilution systems due to high levels of TMP (resultant of hemoconcentration). A mixed dilution technique may be used to address this issue. As further discussed elsewhere herein, mixed dilution is a combination of pre-dilution and post-dilution and as TMP increases the amount of pre-dilution increases and the post-dilution amount is decreased accordingly. However, through the use of two independently supplied dialysate lines, like that of the system described herein, by gradually increasing the ratio of (fresh dialysate supplied to a dialyzer/spent dialysate removed from a dialyzer) as TMP increases, hemoconcentration may be addressed. Without the independently supplied dialysate lines, this cannot be done without balancing issues or some additional process of pre-diluting the blood; however, with the system and techniques described herein, increasing the ratio in a first dialyzer would be offset by decreasing the ratio by an equal amount in a second dialyzer—which results in a net balance of fluid.

Additionally, through the use of independently supplied dialysate lines, the system described herein provides an additional benefit of being able to address secondary membrane formation. Specifically, secondary membrane formation may be addressed in a similar way to that discussed herein in connection with addressing hemoconcentration. A software algorithm may be used in conjunction with the two independently supplied dialysate lines to periodically, throughout treatment (e.g. every 20 minutes), temporarily increase the ratio of fresh/spent dialysate resulting in a net movement of fluid from the dialysate side to the blood side of the dialyzer. By doing this, the membrane formation would be reduced each time the algorithm occurred. It is noted that secondary membrane formation is predominately a problem for hemofiltration, and is less problematic for hemodialysis, but may still be a concern for hemodiafiltration methods.

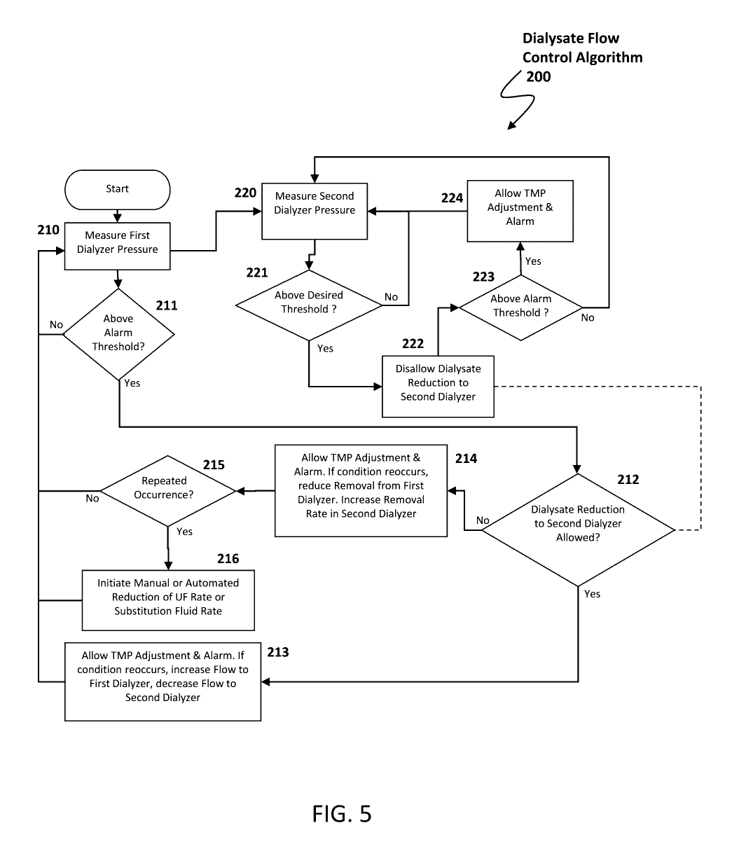

FIG. 5 illustrates a flow diagram 200 showing processing in connection with specific monitoring and control actions taken by a control unit of the hemodiafiltration system according to an embodiment of the system described herein. At a step 210, the transmembrane pressure is measured in the first dialyzer. The process then proceeds to a decision step 211, where transmembrane pressure is analyzed to ensure it is not above the alarm threshold. Processing after the step 211 is further discussed below.

While the transmembrane pressure is analyzed in the first dialyzer, the process is also analyzing the transmembrane pressure of the second dialyzer in a step 220. After the step 220, the process proceeds to a decision step 221 where transmembrane pressure is analyzed to ensure it is not above a predetermined, desired threshold. The desired pressure threshold is a pressure value below a patient safety alarm threshold value, used to prevent further progression towards the safety alarm threshold. If the transmembrane pressure measured at the second dialyzer exceeds this desired pressure threshold (“Yes” at the step 221), the process proceeds to a step 222 where an action is taken to disallow dialysate reduction in the second dialyzer. This impacts the decision made after the process proceeds from step 211 to 212 and shown schematically in the figure with a dashed line.

At a step 212, a decision is made whether it is allowable to adjust the fresh dialysate valves to ameliorate increasing transmembrane pressure based on the outcome of step 222. If dialysate reduction in the second dialyzer is allowed, as would be the case in normal operation, the process proceeds to step 213 and the machine will alarm due to the alarm state. TMP adjustment will be allowed. If, however, the process continues to repeat step 213 more than a pre-determined desired frequency, the machine may take further action and increase fresh dialysate flow to the first dialyzer through increasing the amount of time the valve associated with said fresh dialysate line is opened during software duty cycling. As a result, fresh dialysate flow to the second dialyzer will be decreased by an amount equal to the increase of dialysate flowrate seen in the first dialyzer. In either case, after completing step 213 the process returns to step 210 and again measures transmembrane pressure in the first dialyzer.

If the process reaches the step 212 and dialysate reduction to the second dialyzer is not allowed, the process proceeds to a step 214. At the step 214, like in the step 213, the machine will alarm due to the alarm state and TMP adjustment will be allowed as before. If, however, the process continues to repeat the step 214 more than a pre-determined desired frequency, in the step 214 the amount of spent dialysate fluid removed from the first dialyzer is decreased through decreasing the amount of time the valve associated with said spent dialysate line is opened during software duty cycling. As a result, spent dialysate flow from the second dialyzer will be increased by an amount equal to the decrease in spent dialysate flow seen in the first dialyzer. After completing the step 214, the process proceeds to a decision step 215 where a check is made that this alarm condition is not occurring repeatedly. If the alarm is not repeatedly occurring, the process returns to the step 210, otherwise the process proceeds to a step 206 where the machine takes further corrective action.

At a step 216, an action is required to either reduce the ultrafiltration rate or substitution fluid rate. The action can either be taken manually, through notification to the machine operator to take an action, or automatically through reduction of the ultrafiltration and/or substitution fluid rate through the control unit. After completing the step 216 process returns to the step 210 and again measures transmembrane pressure in the first dialyzer.

As in the first dialyzer, the second dialyzer is also checked to ensure that transmembrane pressure does not exceed an alarm threshold. This is accomplished at a decision step 223 where the transmembrane pressure is compared to an alarm threshold. If the machine is in an alarm-state, the process will move to a step 224 where the machine will alarm and TMP adjustment will be allowed. In this case, or in the case of no alarm state being detected, the process returns to step 220 and again measures pressure in the second dialyzer.

Through this process, transmembrane pressure is maintained at a safe level throughout treatment, and the machine reduces ultrafiltration rate and substitution fluid rate only as a last result resultant of high pressures seen in both the first and second dialyzers.

Various embodiments discussed herein may be combined with each other in appropriate combinations in connection with the system described herein. Additionally, in some instances, the order of steps in the flow diagrams, flowcharts and/or described flow processing may be modified, where appropriate. Further, various aspects of the system described herein may be implemented using software, hardware, a combination of software and hardware and/or other computer-implemented modules or devices having the described features and performing the described functions. The system may further include a display and/or other computer components for providing a suitable interface with a user and/or with other computers.

Software implementations of aspects of the system described herein may include executable code that is stored in a computer-readable medium and executed by one or more processors. The one or more processors may be specific processors configured to control medical devices, such as dialysis machines and/or hemodiafiltration devices. The computer-readable medium may include volatile memory and/or non-volatile memory, and may include, for example, a computer hard drive, ROM, RAM, flash memory, portable computer storage media such as a CD-ROM, a DVD-ROM, an SD card, a flash drive or other drive with, for example, a universal serial bus (USB) interface, and/or any other appropriate tangible or non-transitory computer-readable medium or computer memory on which executable code may be stored and executed by a processor. The system described herein may be used in connection with any appropriate operating system.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A hemodiafiltration device, comprising:
at least two dialyzers for performing hemodiafiltration;
at least one dialysate supply for supplying dialysate;
a sterility filter for generating a sterile substitution fluid; and
a control unit which controls medical fluid inputs and outputs to and from each of the at least two dialyzers, wherein the control unit controls the supply of dialysate such that the dialysate is independently supplied to the at least two dialyzers from the at least one dialysate supply.

2. The hemodiafiltration device according to claim 1, wherein the at least one dialysate supply includes a plurality of dialysate supply lines.

3. The hemodiafiltration device according to claim 1, wherein the at least one dialysate supply includes a plurality of dialysate return lines.

4. The hemodiafiltration device according to claim 1, wherein the medical fluid includes the dialysate, the substitution fluid and/or blood.

5. The hemodiafiltration device according to claim 1, further comprising:
at least one pump configured to pump the medical fluid.

6. The hemodiafiltration device according to claim 5, wherein the at least one pump is controlled by the control unit.

7. The hemodiafiltration device according to claim 1, wherein the control unit includes at least one specific processor configured to control the hemodiafiltration device and that executes executable code stored on a non-transitory computer-readable medium.

8. A method for performing hemodiafiltration, comprising:
performing hemodiafiltration using at least two dialyzers;
supplying dialysate from at least one dialysate supply;
generating a sterile substation fluid using a sterility filter; and
controlling medical fluid inputs and outputs to and from each of the at least two dialyzers using a control unit, wherein the supply of dialysate is controlled such that the dialysate is independently supplied to the at least two dialyzers from the at least one dialysate supply.

9. The method according to claim 8, wherein the at least one dialysate supply includes a plurality of dialysate supply lines.

10. The method according to claim 8, wherein the at least one dialysate supply include a plurality of dialysate return lines.

11. The method according to claim 8, wherein the medical fluid includes the dialysate, the substitution fluid and/or blood.

12. The method according to claim 8, further comprising:
at least one pump configured to pump the medical fluid.

13. The method according to claim 12, wherein the at least one pump is controlled by the control unit.

14. The method according to claim 8, wherein the control unit includes at least one specific processor configured to control the hemodiafiltration device and that executes executable code stored on a non-transitory computer-readable medium.

15. A system for processing medical fluid, comprising:
a hemodiafiltration device including:
at least two dialyzers for performing hemodiafiltration;
at least one dialysate supply for supplying dialysate;
a sterility filter for generating a sterile substitution fluid; and
a control unit which controls medical fluid inputs and outputs to and from each of the at least two dialyzers, wherein the control unit controls the supply of dialysate such that the dialysate is independently supplied to the at least two dialyzers from the at least one dialysate supply; and
at least one component for transporting medical fluid to or from the hemodiafiltration device.

16. The system according to claim 15, wherein the at least one dialysate supply includes a plurality of dialysate supply lines.

17. The system according to claim 15, wherein the at least one dialysate supply includes a plurality of dialysate return lines.

18. The system according to claim 15, wherein the medical fluid includes the dialysate, the substitution fluid and/or blood.

19. The system according to claim 18, wherein the at least one pump is controlled by the control unit.

20. The system according to claim 15, wherein the control unit includes at least one specific processor configured to control the hemodiafiltration device and that executes executable code stored on a non-transitory computer-readable medium.
